(19) United States
(12) Patent Application Publication
(10) Pub. No.: US 2015/0265541 A1
(43) Pub. Date: Sep. 24, 2015

(54) Biodegradable Microcapsules Containing Filling Material

(71) Applicant: AKINA, INC., West Lafayette, IN (US)

(72) Inventors: Kinam Park, West Lafayette, IN (US); Christopher A. Rhodes, New York, NY (US)

(21) Appl. No.: 14/429,304

(22) PCT Filed: Sep. 20, 2013
(86) PCT No.: PCT/US13/60922

§ 371 Date: Mar. 18, 2015

Related U.S. Application Data
(60) Provisional application No. 61/703,723, filed on Sep. 20, 2012.

(51) Int. Cl. A61K 9/50; A61K 31/704; A61K 9/48; A61K 9/00

(52) U.S. Cl. CPC A61K 9/5031; A61K 9/0048; A61K 9/5089; A61K 31/704; A61K 9/4833

(57) ABSTRACT

Biodegradable microcapsules include a biodegradable polymer shell and filling material. The polymer shell completely encompasses the filling material. The filling material may include one or more biodegradable microparticles or a therapeutic agent or both.

### BACKGROUND

Microparticles composed of a biodegradable polymer are useful for controlled release of therapeutic agents. Microfabrication techniques employing templates can be used to produce microparticles having a narrow size distribution. By manipulating microparticle size and composition it is possible to prepare particles with any of a variety of desirable release profiles.

### SUMMARY OF THE INVENTION

Described herein are biodegradable microcapsules containing biodegradable microparticles or a therapeutic agent or both. The microcapsules include a biodegradable polymer shell and filling material. The shell completely encompasses the filling material. The microcapsules can contain one or more microparticles. Thus, the filling material may include one or more microparticles. The biodegradable polymer shell and/or the filling material can optionally include a therapeutic agent. These microparticles can contain one or more therapeutic agents. When the microcapsule contains multiple microparticles the microparticles can be of a single type or of two or more different types. For example, the microcapsule can contain microparticles of two different sizes and/or two different compositions. In some cases each size microparticle is of a different composition. Because the microcapsule can contain microparticles of differing size and composition, it is possible to create microcapsules that contain microparticles having different therapeutic agent release profiles and thus have the ability to release a therapeutic agent over a period of many weeks or months. Thus, the microcapsules can produce consistently controlled levels of drug release and in vivo exposure by providing microcapsules that include particles of two, three or more different release profiles.

Also described are multilayered microcapsules in which the various layers can optionally differ in composition. Such microcapsules can contain a first microparticle that itself contains a second microparticle. In such arrangements the first microparticle essentially acts as a microcapsule or shell for the second microparticle.

The disclosure also features methods for preparing microcapsules and methods for filling microcapsules with one or more components such as microparticles and therapeutic agents.

A microcapsule can be prepared by providing a template having one or more open cavities. A layer of a microcapsule forming composition (e.g., a solution comprising a biodegradable polymer) is coated on the inner surface of the cavities and the composition is allowed to dry thereby forming an open shell or cup. The open shell can then be filled, for example with one or more microparticles or with some other filling material (e.g., a solid, liquid or paste composition containing a therapeutic agent). A composition, which can be the same as that used to coat the inner surface of the cavities, is then applied to seal the core material within the open shell thereby forming a closed shell which completely encloses the filling material thereby forming a microcapsule. The microcapsule is then released from the template.

Described herein is a composition comprising a plurality of microcapsules comprising a shell and filling material, wherein the shell comprises a biodegradable polymer and the filling material comprises at least a first therapeutic agent and the shell completely encloses the filling material. In certain embodiments: the average (on a particle volume basis) Dv (diameter of a spherical particle of the same volume) of the microcapsules is less than 100 μm; the average Dv of the microcapsules is selected from: less than 90, 80, 70, 60 or 50 μm; at least 70% of the microcapsules in the composition vary from the average Dv of the microcapsules in the composition by no more than 50%; the average greatest linear dimension of the microcapsules is selected from: less than 100, 90, 80, 70, 60, 50 or 40 μm; the microcapsules are formulated to release the first therapeutic agent over a period of at least 30 days when injected into a patient; the microcapsules are formulated to release the first therapeutic agent over a period of at least 90 days when injected into a patient; the microcapsules are formulated to release the therapeutic agent over a period of at least 90 days when introduced into an eye of a patient; the microcapsules are formulated to release the therapeutic agent over a period of at least 180 days when injected into an eye of a patient; the shell is an outer shell and the filling material comprises an inner shell comprising a biodegradable polymer that encloses a composition comprising a therapeutic agent; and the composition enclosed by the inner shell comprises microparticles.

The methods describe herein can also be used to make larger microcapsules. For example, microcapsules that have greatest linear dimension of between 0.5 and 10 mm. Thus, the microcapsule can be a cylindrical rod with dimensions of, for example, 2 mm×0.75 mm. In some cases the cylindrical rod has a diameter of less than 100 microns (e.g., 30-100 microns, 75 microns, or 50 microns) and a height of less than 150 microns (e.g., 50-150 microns, 125 microns, 100 microns, 75 microns, or 50 microns). In some cases the greatest linear dimension is less than 300 microns, less than 200 microns or less than 1000 microns. Suitable greatest linear dimensions can be between 500 (400, 300, 200 or 100) microns and 25 microns, 30 microns or 40 microns. Because the particles are formed using a template, a composition comprising the microcapsule can be relatively monodisperse.

The total weight of the microcapsule can be 100 to 5000 micrograms (e.g., 250-1000 micrograms). Such large microcapsules can contain a greater amount of therapeutic agent and the agent can be released over a longer period of time. Thus, a larger microcapsule can release a therapeutic agent over period of at least 6 months, 1 year, 2 years and the various individual components of the microcapsule can release therapeutic agents over a period of 3 months, 6 months, 9 months, 1 year, 18 months, 2 years or longer.

Also described is a composition comprising: a) microcapsules of a first type comprising a shell and filling material, wherein the shell comprises a biodegradable polymer and the filling material comprises a therapeutic agent and wherein the shell completely encloses the filling material; and b) microcapsules of a second type comprising a shell and filling material, wherein the shell comprises a biodegradable polymer and the filling material comprises a therapeutic agent and wherein the shell completely encloses the filling material, wherein the microcapsules of the first type and the microcapsules of the second type differ in one or both of average Dv and composition.

In various embodiments: the microcapsules of the first type are formulated to release the therapeutic agent over a period of at least three months when injected into a patient and the microcapsules of the second type are formulated to release the therapeutic agent over a period of at least six months when injected into a patient; the filling material comprises a plurality of microparticles of a first type, wherein the microparticles of the first type comprise a biodegradable polymer; the filling material further comprises microparticles of a second type, wherein the microparticles of the second type comprise a biodegradable polymer; the microparticles of the first type comprise a therapeutic agent and the microparticles of the second type comprise a therapeutic agent; the microparticles of the first type have a first therapeutic agent release profile and the microparticles of the second type have a second therapeutic agent release profile; the microparticles of the first type release the 90% of their therapeutic agent within 1 to 3 months of exposure to a physiological solution; the microparticles of the second type release the 90% of their therapeutic agent within 3-6 months of exposure to a physiological solution; the first and second therapeutic agents are the same; the first and second therapeutic agents are different; the filling material further comprises microparticles of a third type, wherein the microparticles of the third type comprise a biodegradable polymer; the shell comprises a therapeutic agent; the shell does not comprise a therapeutic agent; the filling material comprises a therapeutic agent that is not in admixture with a biodegradable polymer; and the filling material comprises a polypeptide.

Also disclosed is a method for preparing a microcapsule comprising a shell and filling material, the method comprising: providing a template having at least one cavity; forming a layer of a composition comprising a biodegradable polymer on the surface of the at least one cavity; allowing the composition comprising a biodegradable polymer to solidify thereby forming an open shell; filling the open shell with a core material; sealing the open shell by applying a layer of a composition comprising a biodegradable polymer and allowing the composition comprising the biodegradable polymer to solidifying thereby forming a microcapsule comprising a shell enclosing the core material; and releasing the microcapsule from the template.

In various embodiments of the method: the template comprises a water-soluble polymer; the template comprises a hydrogel; and the composition comprising a biodegradable polymer is a liquid or a paste.

Because the template used to prepare the microcapsules can be formed using any of a variety of microfabrication techniques and can include a plurality of uniformly shaped and uniformly sized cavities, the methods described herein provide a reliable and scalable process that allows fabrication of multifunctional microcapsules and larger implantable structures. The methods described herein enable the fabrication of microcapsules with structures organized in a predefined fashion, i.e., an outer shell of specific thickness and an inner chamber that is filled with filling material containing various components, e.g., two or more different types of microparticles. When the shell is filled with microparticles, the number, size, and arrangement of microparticles can be controlled.

The microcapsule can be filled with a drug in an aqueous or organic composition (e.g., a solution, suspension, paste or gel) or with dry drug powder. If a composition containing a liquid is used to fill the microcapsule, the liquid may be evaporated, leaving a solid material such as a crystalline or amorphous drug. The drug containing solution or drug powder can be present in addition to drug-containing microparticles.

In some cases the material used to form the shell of microcapsule contains a therapeutic agent, and this therapeutic agent can be the same as or different from a therapeutic agent that is within the filling material. In some cases the material used to form the shell of the microcapsule does not contain a therapeutic agent. Because such microcapsules can protect the drug in the core material from immediate release, there may not be a burst drug release from the microcapsules. Alternatively, an outer layer containing drug may be used to provide an initial release if desired for the intended therapeutic purpose.

The microcapsules can be formulated for administration to a patient, for example by injection. The microcapsules can be present in a composition together with one or more pharmaceutically acceptable carriers or excipients.

A wide variety of polymers can be used to form the microcapsule. Non-limiting examples of polymers include: poly(lactic-co-glycolic acid) (PLGA), poly(lactic acid) (PLA), poly(glycolic acid) (PGA), poly(ε-caprolactone), and poly(ortho ester), and other natural biodegradable polymers, such as collagen, chitosan, and poly(amino acid). Combinations of polymers may also be used. Implant shells and filling materials may be prepared from biodegradable polymers listed above or non-biodegradable polymers such as poly ethylene co-vinyl acetate, poly methyl methacrylates, polybutyl methacrylate, poly 1,2 butadiene. Other suitable polymers can include various homopolymers, copolymers, straight, branched-chain, or cross-linked derivatives, e.g., polycarbamates or polyureas, cross-linked poly(vinyl acetate), ethylene-vinyl ester copolymers having an ester content of 4 to 80% such as ethylene-vinyl acetate (EVA) copolymer, ethylene-vinyl hexanoate copolymer, ethylenevinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, an mixtures thereof.

Once formed, the microcapsules can be released from the template by any of a variety of methods. For example, in the case of a template formed of gelatin or another material capable of undergoing a sol-gel transition, e.g., hydrogel templates, the microcapsules can be released by either changing the temperature of the template or placing the template in aqueous solution that can dissolve the template thereby releasing the microcapsules. In other cases the microcapsules are released from the template mechanically while preserving the template.

The microcapsule-forming template can comprise a hydrogel such as, but not limited to, gelatin, poly(acrylic acid), poly(hydroxyethyl methacrylate), poly(vinyl alcohol), dextran, and ethylcellulose.

Other suitable template materials can include a mixture of Pluronics and poly(ethylene glycol) (PEG), water-soluble polymers, such as polyvinylpyrrolidone (PVP) and dextran, and mixtures of water-soluble polymers, such as PVP and PEG.

Microfabrication techniques employing hydrogel templates are described in: Park et al. Journal of Controlled Release 141:314-319. Other microfabrication techniques employing other types of templates are described in Whitesides et al. 2001 Annual Review Biomed Engineering 3:335-73.

The template can be formed using a mold, for example, prepared by coating a silicon wafer with photoresist and etching out the desired shape for the template. The template is formed on the resulting mold. The cavities in the template may be any desired shape such that the resulting microcapsules can have at least one cross-section that is square, rectangular, round or some other desired shape.

The shells and the microparticles are generally substantially uniform mass and are substantially monodisperse in shape, surface area, height and mass. For example, in a population of particles (for example a population contained in single dose of a pharmaceutical composition), as few as 1% or less of the particles vary from the average greatest linear dimension by more than 15% (e.g., few than 5% or the particles vary from the average greatest linear dimension by 5, 6, 7, 8, 9. to 10 microns.

### DRAWINGS

FIGS. 1A-1D are photographs of 50 μm diameter microcapsules containing microparticles. (FIG. 1A) Microcapsule loaded with blue fluorescent beads (5.5 μm diameter); (FIG. 1B & FIG. 1C) Microcapsule loaded with red and blue fluorescent beads (10 μm and 5.5 μm diameters, respectively); (FIG. 1D) Microcapsule loaded with red, green, and blue fluorescent beads (10 μm, 15 μm, and 5.5 μm diameters, respectively). Scale bars correspond to 25 μm.

FIGS. 1E-1H are fluorescent images of microcapsule containing blue, green, and red fluorescent beads (of ˜5.5 μm diameters) in a series of orientations demonstrating the presence of the fluorescent beads in its core: (FIG. 1E) Top view along z-axis; (FIG. 1F) Side view along y-axis; (FIG. 3G) Side view at 45° angle, and (FIG. 3H) Side view along x-axis. The diffused light around the fluorescent beads is due to the scattering and reflection of fluorescent light in the PLGA matrix.

FIG. 2 is a photograph of microcapsules with spatially predefined zones fabricated by hydrogel template strategy. The microcapsules have a PLGA shell containing blue microparticles and inner core containing red microparticles.

### DETAILED DESCRIPTION

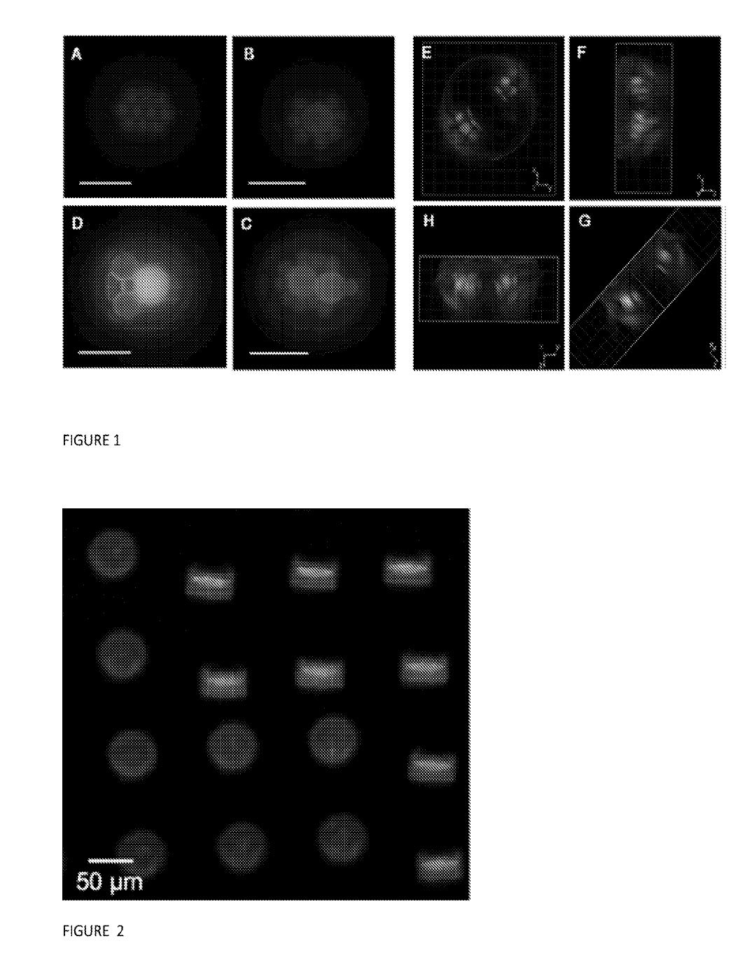

FIG. 1 schematically depicts a microcapsule containing a number of different microparticles. Each particle may consist of a formulation of drug designed for a specific release profile, varying from essentially immediate release to extended release. Each particle formulation may contain a biodegradable polymer and a first drug alone or in combination with one or more of: a stabilizer, an excipient (e.g., an excipient that decreases release rate or an excipient that increases release rate), a second drug, an additive (e.g., an additive that increase or decrease release rates of the surrounding polymer systems, increase or decrease the water content, increase or decrease the pH of the surrounding environment). Where two or more different types of microparticles are present they can be composed of biodegradable polymers that differ in chemical composition, molecular weights, crystallinity, or other factors.

As show in FIG. 2, by preparing microcapsules containing various forms of a drug, it is possible to prepare microcapsules that release the drug over many weeks or months and sustain the drug concentration at or above the expected therapeutic for an extended period. For example, the first form is a formulation of drug designed for immediate release upon injection, e.g., native drug alone in particle suspension medium or drug formulated into a fast-releasing system that may or may not contain polymer. The second form is a PLGA microparticle formulation of the same drug having a common PLGA release profile—initial release, a lag phase, and extended release phase lasting one to three months. The third form is a PLGA microparticle similar to the microparticle just described that is encapsulated in an outer layer of a slower release polymer such as PLA or polycaprolactone. This outer layer degrades over a period of three to twelve months releasing the inner PLGA microparticle which in turn degrades over an additional few months. The resulting PK profile is a combination of the three drug release profiles resulting in exposure above the therapeutic level for six to twelve months.

Among the therapeutic agents that can be incorporated into a microcapsule or into the filling material within a microcapsule (e.g., a microparticle) including, but are not limited to, small molecule drugs, peptide drugs, protein drugs, oligonucleotides, antibodies.

A variety of different polymers can be used in the microparticles, including, but not limited to, biodegradable polymers, non-biodegradable polymers, polymers of naturally derived materials, natural biopolymers, polymers that form hydrogels, and thermo-reversible polymers. Examples of useful polymers include, but are not limited to: poly(acrylic acid); poly(methacrylic acid); poly(hydroxyl acid); PLA; PGA; PLGA; polyanhydride; polyorthoester; polyamide; polycarbonate; polyalkylene; polyethylene; polypropylene; polyalkylene glycol; poly(ethylene glycol); poly(alkylene oxide); poly(ethylene oxide); poly(alkylene terephthalate); poly(ethylene terephthalate); poly(vinyl alcohol), polyvinyl ether; polyvinyl ester; polyvinyl halide; poly(vinyl chloride); polyvinylpyrrolidone; polysiloxane; poly(vinyl acetate); polyurethane; co-polymer of polyurethane; derivativized cellulose; alkyl cellulose; hydroxyalkyl cellulose; cellulose ether; acellulose ester; nitro cellulose; methyl cellulose; ethyl cellulose; hydroxypropyl cellulose; hydroxyl-propyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobuyl acrylate); polyoctadecyl acrylate); poly(butyric acid); poly(valeric acid); poly(lactide-co-caprolactone); a copolymer of poly(lactide-co-caprolactone); a blend of poly(lactide-co-caprolactone); polygalactia; poly-(isobutyl cyanoacrylate); poly(2-hydroxyethyl-L-glutamine); poly(DL-lactide-co-ε-caprolactone) (DLPLCL);, collagen; gelatin; agarose; gelatin/ε-caprolactone; collagen-GAG; fibrin, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, poly-carbonates, poly(ethylene oxide), polymers of acrylic acid cross-linked with polyalkenyl ethers or divinyl glycol (e.g. CARBOPOL® 934P, 71G, 971P, 974P), silicone polymer; hyaluronan gel, PEG-PLGA-PEG triblock copolymer, RESOMER RGP t50106 (Boehringer Ingelheim); ReGel (MacroMed Incorporated), ABA-type or BAB-type triblock copolymers or mixtures thereof, biodegradable, hydrophobic A polymer block comprising a polyester or poly(orthoester), in which the polyester is synthesized from monomers (e.g., selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons), glycerin-based gels, glycerin-derived compounds, conjugated, or crosslinked gels, matrices, hydrogels, and polymers, alginates, and alginate-based gels, native and synthetic hydrogel and hydrogel-derived compounds; alginate hydrogels SAF®-Gel (ConvaTec, Princeton, N.J.), Duoderm Hydroactive Gel (ConvaTec), Nu-gel® (Johnson & Johnson Medical, Arlington, Tex.); Carrasyn®(V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). Co-polymers of these various polymers can also be used.

### Examples

### Materials & Methods

The experiments were performed using commercially available materials: gelatin, poly(vinyl alcohol), polyvinylpyrrolidone, dextran, and ethylcellulose (Sigma), poly(lactic-co-glycolic acid) (PLGA, Akina and Lactel) of different molecular weights (MW 36,000, IV 0.7 dL/g; MW 65,000, IV 0.82 dL/g; MW 112,000, IV 1.3 dL/g) were used in our experiments. Fluorescent microbeads were purchased from Bangs laboratories. Quantum dots were purchased from Aldrich.

### 1. Fabrication of Silicon Master Templates by E-Beam Lithography

Circular patterns for 500 mil diameter were designed using Auto CAD 2007 program. A 3″ silicon wafer (100) covered with 1 μm thick SiO2 layer (University Wafer) was spin coated with poly(methyl methacrylate) (PMMA, Microchem) photoresist of 300 nm thick layer using a spin coated (SCS P6708 spin coating system, 3500 rpm, 30 sec). The coated PMMA photoresist layer was exposed to electron beam (e-beam) in a preprogrammed pattern using Leica VB6 High Resolution Ultrawide Field Photolithography Instrument (operating at 100 KV, transmission rate 25 MHz current 5 nA). After e-beam lithography, the silicon wafer was developed in 3:1 isopropanol:methyl isobutyl ketone solution to remove exposed regions of the photoresist. A 5 nm chromium layer and 20 nm gold layer were deposited on to this pattern followed by liftoff of the residual PMMA film in refluxing acetone. The pattern was transferred to the underlying silicon oxide by deep reactive ion etching with SF6/O2 plasma. The generated silicon master template was used in the fabrication of hydrogel templates.

### 2. Fabrication of Silicon Wafer Master Templates by Photolithography

A silicon wafer was spin coated with SU8 2010 photoresist (Microchem, MA) at 3,500 rpm for 30 sec to obtain a desired thickness followed by baking at 95° C. for 3 min. The photoresist coated silicon wafer was exposed to UV radiation through a mask containing 10 μm diameter circular pattern for 12 sec. After exposure, the silicon wafer was post baked at 95° C. for 3 min followed by development in SU-8 developer for 2 min. The silicon wafer was rinsed with isopropanol and dried with nitrogen gas. The wafer thus fabricated contained wells with diameter ranging from 1.5 μm to 50 um or larger.

### 3. Fabrication of Dissolvable Templates

Temporary templates for producing microcapsules can be made by polymers that can be dissolved in aqueous solution or in a mixture of aqueous and organic solutions (e.g., water and ethanol). The temperature can be altered, either increased or decreased from the room temperature, to dissolve a temporary template. Alternatively, pH of aqueous solution can be changed for dissolving a temporary template. A clear gelatin solution (30% w/v in water, 10 ml) at 50° C. was transferred with a pipette onto a silicon wafer master template, or an optional intermediate template made of poly(dimethyl siloxane) (PDMS), (3″ diameter) containing circular pillars (e.g., of 10 μm diameter and 10 μm height). The gelatin solution was evenly spread to form a thin film completely covering the PDMS template and cooled to 4° C. for 5 min by keeping it in a refrigerator. Cooling resulted in the formation of an elastic and mechanically strong gelatin template. After cooling, the gelatin template was peeled away from the PDMS template. The obtained gelatin template was ˜3″ in diameter, contained circular wells (e.g., of 10 μm diameter and 10 μm depth). The gelatin template was examined under a bright field reflectance microscope to determine its structural integrity.

A clear poly(vinyl alcohol) (PVA) solution (15% w/v in water, 5 ml) was transferred with a pipette onto a PDMS template (3″ diameter) containing circular pillars (e.g., of 10 μm diameter and 10 μm height). The PVA solution was evenly spread to form a thin film completely covering the PDMS template and kept in an oven at 70° C. for 30 min. This step resulted in the formation of a thin and mechanically strong PVA template. The PVA template was peeled away from the PDMS template. The obtained PVA template was ˜3″ in diameter, contained circular wells (e.g., of 10 μm diameter and 10 μm depth). The PVA template was examined under a bright field reflectance microscope to determine its structural integrity.

A clear polyvinylpyrrolidone (PVP) solution (7.5% w/v in water, 5 ml) was transferred with a pipette onto a PDMS template (3″ diameter) containing circular pillars (e.g., of 10 μm diameter and 10 μm height). The PVP solution was evenly spread to form a thin film completely covering the PDMS template and kept in an oven at 70° C. for 30 min. This step resulted in the formation of a thin and mechanically strong PVP template. The PVP template was peeled away from the PDMS template. The obtained PVP template was ˜3″ in diameter, contained circular wells (e.g., of 10 μm diameter and 10 μm depth). The PVP template was examined under a bright field reflectance microscope to determine its structural integrity.

A clear dextran solution (10% w/v in water, 5 ml) was transferred with a pipette onto a PDMS template (3″ diameter) containing circular pillars (e.g., of 10 μm diameter and 10 μm height). The dextran solution was evenly spread to form a thin film completely covering the PDMS template and kept in an oven at 70° C. for 30 min. This step resulted in the formation of a thin and mechanically strong dextran template. The dextran template was peeled away from the PDMS template. The obtained dextran template was ˜3″ in diameter, contained circular wells (e.g., of 10 μm diameter and 10 μm depth). The dextran template was examined under a bright field reflectance microscope to determine its structural integrity.

A clear ethylcellulose solution (10% w/v in water, 5 ml) was transferred with a pipette onto a PDMS template (3″ diameter) containing circular pillars (e.g., of 10 μm diameter and 10 μm height). The ethyl cellulose solution was evenly spread to form a thin film completely covering the PDMS template and kept in an oven at 70° C. for 30 min. This step resulted in the formation of a thin and mechanically strong ethyl cellulose template. The ethyl cellulose template was peeled away from the PDMS template. The obtained ethylcellulose template was ˜3″ in diameter, contained circular wells (e.g., of 10 μm diameter and 10 μm depth).

4. Microcapsules Filled with Fluorescent Beads

Briefly, 100 μl of 10% PLGA (MW 65,000, IV 0.82 dL/g) solution w/v in dichloromethane was transferred with a pipette onto a 3″ diameter hydrogel template containing circular wells of 50 μm diameter and depth. The PLGA solution was evenly spread on the hydrogel template followed by evaporation of CH₂Cl₂ (10 min, room temperature). This step resulted in the formation of cup-shaped microstructures in the gelatin template. In the second step, the PLGA-covered wells in the gelatin template were filled with 30 μl of an aqueous suspension of fluorescent microspheres (glacial blue, 5.5 μm diameter). The gelatin template was then left at room temperature for 10 min followed by flushing with a gentle stream of nitrogen gas to remove the water from the wells. Finally, 100 μl of PLGA solution (MW 65,000, IV 0.82 dL/g) was transferred onto the gelatin template, followed by spreading it evenly on the template. This step resulted in the closing of the PLGA cups filled with fluorescent microspheres. The gelatin template was dissolved in water to obtain free microcapsules containing fluorescent microspheres. The obtained microcapsules were characterized by bright field and fluorescence microscopy.

5. Microcapsules Filled with Red, Green, and Blue Fluorescent Beads

Microcapsules containing red, blue, and green fluorescent beads were fabricated by performing the experimental procedure #4 above. In this experiment, a mixture of red, green, and blue fluorescent beads was used.

6. Microcapsules with Blue Quantum Dots in the Shell and Red Quantum Dots in the Core

Briefly, 100 μm of 10% PLGA (MW 65,000, IV 0.82 dL/g) solution w/v in dichloromethane containing 25 μm of red quantum dots (20 nm diameter) was transferred with a pipette onto a 3″ diameter hydrogel template containing circular wells of 50 μm diameter and depth, respectively. The PLGA solution was evenly spread on the hydrogel template followed by evaporation of CH₂Cl₂ (10 min, room temperature). This step resulted in the formation of cup-shaped microstructures in the gelatin template. In the second step, the PLGA-covered wells in the gelatin template were filled with 100 μm of 20% PLGA (MW 65,000, IV 0.82 dL/g) solution w/v in dichloromethane containing 25 μm of blue quantum dots (20 nm diameter). Finally, 100 μm of 10% PLGA (MW 65,000, IV 0.82 dL/g) solution w/v in dichloromethane containing 25 μl of red quantum dots (20 nm diameter) was transferred onto the gelatin template, followed by spreading it evenly on the template. This step resulted in the closing of the PLGA cups filled with fluorescent microspheres. The gelatin template was dissolved in water to obtain free microcapsules containing fluorescent microspheres. The obtained microcapsules were characterized by bright field and fluorescence microscopy.

7. Microcapsules with In Situ Crystallizable Doxorubicin Drug in the Core

First, 100 μl of 10% PLGA (MW 65,000, IV 0.82 dL/g) solution w/v in dichloromethane was transferred with a pipette onto a 3″ diameter hydrogel template containing circular wells of 50 μm diameter and depth, respectively. The PLGA solution was evenly spread on the hydrogel template followed by evaporation of CH₂Cl₂ (10 min, room temperature). This step resulted in the formation of cup-shaped microstructures in the gelatin template. In the second step, the PLGA-covered wells in the gelatin template were filled with 30 μl of doxorubicin solution in methanol (1 mg/ml). The gelatin template was then left at room temperature for 15 min to let the formation of doxorubicin crystals in the wells. This step was followed by gently flushing with a stream of nitrogen gas to completely remove methanol. Finally, 100 μl of PLGA solution (MW 65,000, IV 0.82 dL/g) was transferred onto the gelatin template, followed by spreading it evenly on the template. This step resulted in the closing of the PLGA cups filled with fluorescent microspheres.

### 8. Fabrication of Lithium Iron Phosphate Microcylinders

First, 250 μl of lithium iron phosphate (LiFePO4) slurry in toluene (250 mg/ml) was transferred with a pipette onto a 3″ diameter PVP template containing circular wells of 50 μm diameter and depth, respectively. The slurry was evenly spread on the PVP template. This filled template was kept in the oven (80° C., 5 h). This step resulted in the formation of solid LiFePO4 microcylinders in the PVP template.

### 9. Collection of Free Microcapsules

Gelatin templates filled with quantum dot/PLGA solution were left at room temperature for 10 min to ensure that all CH₂Cl₂ solvent has been evaporated from the templates. A batch of 10 gelatin templates were dissolved in a 100 ml beaker containing 50 ml of Nanopure water at 40° C. and gently shaken for 2 min to completely dissolve the templates. This step resulted in complete release of the free microcapsules into the solution. The solution was transferred into conical tubes (15 ml) and centrifuged for 5 min (Eppendorf Centrifuge 5804, Rotor A-4-44, at 5,000 rpm, 19.1 RCF). The pellet obtained upon centrifugation was freeze dried and stored in a refrigerator. This pellet upon resuspension in 1 ml of Nanopure water formed free and isolated microcapsule dispersion. The PVP/PEG templates were dissolved in water at room temperature to collect the formed microcapsules. The main advantage of the PVP/PEG template over others is that it can be dissolved in water at room temperature or at lower temperatures, allowing flexibility in collecting the microcapsules that contain temperature sensitive drugs, such as protein drugs and antibodies.

### 10. Characterization of Polymer Microstructures

The polymer microstructures were characterized by bright field, confocal fluorescence imaging and scanning electron microscopy. Bright field and confocal fluorescence imaging was performed on an Olympus Spinning Disc Confocal Imaging Microscope BX61-DSU equipped with Intelligent Imaging Innovations Slide Book 4.0 software for automated Z-stack and 3-D image analysis. Scanning electron microscopy was performed on FEI NOVA nano SEM and Hitachi 4800 SEM.

The microcapsules described above are PLGA have a 50 μm diameter and were filled with beads of different fluorescent colors. Microcapsules having other sizes can be be made by a similar process. The microcapsules filled with blue fluorescent beads (5.5 μm diameter) clearly indicate that the beads are present in the core of the microcapsule (FIG. 2). The ability to mix different filling material is demonstrated by the filling microcapsules with a mixture of fluorescent beads. The microcapsules were filled with blue fluorescent beads (FIG. 2A) red and blue fluorescent beads (FIG. 3B and FIG. 3C), and also blue, green, and red fluorescent beads (FIG. 3D). Importantly, the fluorescent beads are placed in the core of the capsule (FIG. 3E, FIG. 3F, FIG. 3G and FIG. 3H). The diffused light around the fluorescent beads is a result of the reflection and scattering of the fluorescent light in the PLGA layers of the matrix. From the positioning of the beads in the core of the microcapsule, one can envision fabrication of multicomponent nano- and microdevices. Microcapsules filled with different mixtures of fluorescently labeled beads are useful as markers.

What is claimed is:

1. A composition comprising a plurality of microcapsules comprising a shell and filling material, wherein the shell comprises a biodegradable polymer and the filling material comprises at least a first therapeutic agent and the shell encloses the filling material.

2. The composition of claim 1 wherein the average Dv of the microcapsules is less than 100 μm.

3. The composition of claim 2 wherein the average Dv of the microcapsules is selected from: less than 90, 80, 70, 60 or 50 μm.

4. The composition of claim 2 wherein at least 70% of the microcapsules in the composition vary from the average Dv of the microcapsules in the composition by no more than 50%.

5. The composition of claim 1 or claim 2 wherein the average greatest linear dimension of the microcapsules is selected from: less than 100, 90, 80, 70, 60, 50 or 40 μm.

6. The composition of claim 1 wherein the microcapsules are formulated to release the first therapeutic agent over a period of at least 30 days when introduced into or around the eye of a patient.

7. The composition of claim 1 wherein the microcapsules are formulated to release a therapeutic agent over a period of at least 90 days when introduced into or around the eye of a patient.

8. The composition of claim 1 wherein the microcapsules are formulated to release the therapeutic agent over a period of at least 90 days when introduced into or around the eye of a patient.

9. The composition of claim 8 wherein the microcapsules are formulated to release the therapeutic agent over a period of at least 180 days when introduced into or around the eye of a patient.

10. The composition of claim 1 wherein the shell is an outer shell and the filling material comprises an inner shell comprising a biodegradable polymer that encloses a composition comprising a therapeutic agent.

11. The composition of claim 10 wherein the composition enclosed by the inner shell comprises microparticles comprising a biodegradable polymer.

12. The composition of claim 1 comprising:
a) microcapsules of a first type comprising a shell and filling material, wherein the shell comprises a biodegradable polymer and the filling material comprises a therapeutic agent and wherein the shell completely encloses the filling material; and
b) microcapsules of a second type comprising a shell and filling material, wherein the shell comprises a biodegradable polymer and the filling material comprises a therapeutic agent and wherein the shell completely encloses the filling material,
wherein the microcapsules of the first type and the microcapsules of the second type differ in one or both of average Dv and composition.

13. The composition of claim 12 wherein microcapsules of the first type are formulated to release the therapeutic agent over a period of at least three months when injected into a patient and the microcapsules of the second type are formulated to release the therapeutic agent over a period of at least six months when injected into a patient.

14. The composition of claim 1 wherein the filling material comprises a plurality of microparticles of a first type, wherein the microparticles of the first type comprise a biodegradable polymer.

15. The composition of claim 14 wherein the filling material further comprises microparticles of a second type, wherein the microparticles of the second type comprise a biodegradable polymer.

16. The composition of claim 15 wherein the microparticles of the first type comprise a therapeutic agent and the microparticles of the second type comprise a therapeutic agent and both the therapeutic agent and the biodegradable polymer can be the same or different.

17. The composition of claim 16 wherein the microparticles of the first type have a first therapeutic agent release profile and the microparticles of the second type have a second therapeutic agent release profile.

18. The composition of claim 17 wherein the microparticles of the first type release the 90% of their therapeutic agent within 1 to 3 months of exposure to a physiological solution or a patient.

19. The composition of claim 17 wherein the microparticles of the second type release the 90% of their therapeutic agent within 3-6 months of exposure to a physiological solution or a patient.

20. The composition of claim 16 wherein the first and second therapeutic agents are the same.

21. The composition of claim 16 wherein the first and second therapeutic agents are different.

22. The composition of claim 15 wherein the filling material further comprises microparticles of a third type, wherein the microparticles of the third type comprise a biodegradable polymer.

23. The composition of claim 1 wherein the shell comprises a therapeutic agent.

24. The composition of claim 1 wherein the shell does not comprise a therapeutic agent.

25. The composition of claim 1 wherein the filling material comprises a therapeutic agent that is not in admixture with a biodegradable polymer.

26. The composition of claim 1 wherein the filling material comprises a polypeptide.

27. A method for preparing a microcapsule comprising a shell and filling material, the method comprising:
providing a template having at least one cavity;
forming a layer of a composition comprising a biodegradable polymer on the surface of the at least one cavity by applying a liquid of gel composition comprising a biodegradable polymer to at least on cavity;
allowing the composition comprising a biodegradable polymer to solidify thereby forming an open shell;
filling the open shell with a core material;
sealing the open shell by applying a liquid or gel composition comprising a biodegradable polymer comprising a biodegradable polymer to the opening of the shell and allowing the liquid or gel composition comprising the biodegradable polymer to solidifying thereby forming a microcapsule comprising a shell enclosing the core material; and
releasing the microcapsule from the template.

28. The method of claim 27 wherein the template comprises a water-soluble polymer.

29. The method of claim 27 wherein the template comprises a hydrogel.

30. The method of claim 27 wherein the composition comprising a biodegradable polymer is a liquid or a paste.

31. The method of claim 1 wherein the shell comprises a water impermeable polymer membrane, a semi-permeable membrane, a biodegradable polymer in combination with a water impermeable polymer membrane or a water impermeable membrane.

32. The method of claim 1 wherein all therapeutic agent is released over a period within 120 days.
